(19) United States
(12) Patent Application Publication
(10) Pub. No.: US 2025/0259746 A1
(43) Pub. Date: Aug. 14, 2025

(54) MENOPAUSE PREDICTING TOOL

(71) Applicants: Kiran Kumar, La Jolla, CA (US); Philip Tajanko, La Jolla, CA (US)

(72) Inventors: Kiran Kumar, La Jolla, CA (US); Philip Tajanko, La Jolla, CA (US)

(21) Appl. No.: 19/049,414

(22) Filed: Feb. 10, 2025

Related U.S. Application Data
(60) Provisional application No. 63/551,705, filed on Feb. 9, 2024.

(51) Int. Cl. G16H 50/20

(52) U.S. Cl. CPC G16H 50/20

(57) ABSTRACT

An Artificial Intelligence (AI) system trained by a plurality of training data. The AI system is trained by relationships between a time to final menstrual period and ratios between follicle-stimulating hormone levels and estradiol levels, race, cholesterol levels, and a presence of one or more contraceptives. The AI system is configured to accept data from only one sample from a user as input and generate a prediction of a time to final menstrual period for the user as output.

### CROSS-REFERENCE TO RELATED APPLICATIONS

This application is a non-provisional and claims benefit of U.S. Provisional Application No. 63/551,705 filed Feb. 9, 2024, the specification of which is incorporated herein in its entirety by reference.

### FIELD OF THE INVENTION

The present invention is directed to a menopause predicting algorithm which uses test results from a single blood draw.

### BACKGROUND OF THE INVENTION

Women are being completely blindsided by perimenopause and menopause as they enter the transition uninformed and unprepared. 31% are wrongly diagnosed, and 45% are not treated despite wanting treatment. This results in mental whiplash and an inability to plan & prepare. Furthermore, this results in a healthcare burden of $660 billion. Thus, there exists a present need for a menopause predicting algorithm which uses test results from a single blood draw.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a flow chart of a method for training and developing the AI system of the presently claimed invention.

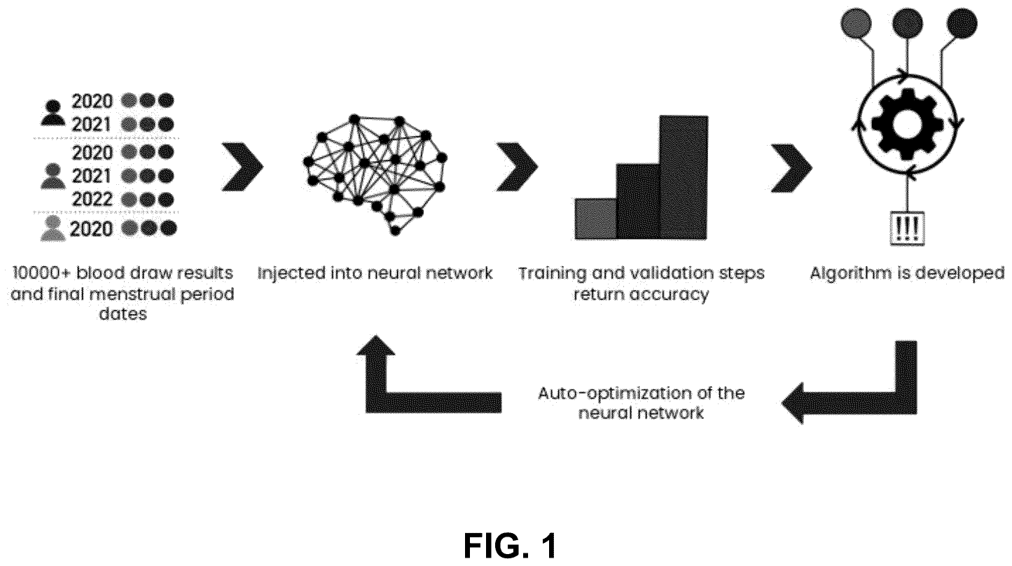

### DETAILED DESCRIPTION OF THE INVENTION

In some embodiments, the present invention features an Artificial Intelligence (AI) system trained by a plurality of training data comprising relationships between a time to final menstrual period and ratios between follicle-stimulating hormone levels and estradiol levels, race, cholesterol levels, and a presence of one or more contraceptives, configured to accept data from only one sample from a user as input and generate a prediction of a time to final menstrual period for the user as output.

The sample may be collected from an at-home test kit. The sample may comprise a blood sample. The training data may comprise 10000+ blood draw results and final menstrual period dates. The AI model may be auto-optimized.

For final menstrual period dates, the highest weight of predictive values is the ratio between FSH and estradiol. This allows for the use of just one blood reading to make an accurate prediction. In some embodiments, the AI model may additionally accept data on the user's ovarian cycle to increase accuracy. In some embodiments, the training data may comprise data on patients that have not yet had a final menstrual event. To overcome this, a Kapplen-Meir estimator is used to fill in the blanks and estimate the final menstrual period, thus allowing this to still be used as training data.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

Reference numbers recited herein, in the drawings, and in the claims are solely for ease of examination of this patent application and are exemplary. The reference numbers are not intended in any way to limit the scope of the claims to the particular features having the corresponding reference numbers in the drawings.

What is claimed is:

1. An Artificial Intelligence (AI) system trained by a plurality of training data comprising relationships between a time to final menstrual period and ratios between follicle-stimulating hormone levels and estradiol levels, race, cholesterol levels, and a presence of one or more contraceptives, configured to accept data from only one sample from a user as input and generate a prediction of a time to final menstrual period for the user as output.
